(19) United States
(12) Patent Application Publication
(10) Pub. No.: US 2003/0087277 A1
(43) Pub. Date: May 8, 2003

(54) Means and methods for detection of binding of members of specific binding pairs

(76) Inventors: Wolfgang Fritzsche, Jena (DE); Andrea Czaki, Camburg (DE); Johann Michael Koehler, Golmsdorf (DE); Robert Moeller, Jena (DE); Frederik Schut, Den Haag (NL); Louis Oosting, Groningen (NL); Paris Som Tjwan Tan, Haren (NL)

Correspondence Address: JORDAN AND HAMBURG LLP, 122 EAST 42ND STREET, SUITE 4000, NEW YORK, NY, 10168, US

(21) Appl. No.: 10/215,789

(22) Filed: Aug. 9, 2002

Related U.S. Application Data
(63) Continuation-in-part of application No. 09/869,206, filed on Jun. 25, 2001.
Application No. 09/869,206 is a 371 of international application No. PCT/EP99/10334, filed on Dec. 22, 1999.

(30) Foreign Application Priority Data
Dec. 23, 1998 (DE) 198 60 547

(51) Int. Cl. C12Q 1/68; C12M 1/34

(52) U.S. Cl. 435/6; 435/287.2

(57) ABSTRACT

The present invention relates to an affinity sensor and methods suitable for use in an affinity sensor for detecting specific molecular binding events, as is particularly used in the molecular biological field, for example, in the medical diagnostics, in the biosensor technology or in the DNA-microarray technology, and application of the same. A method for detecting binding of members of a specific binding pair of the invention comprises providing a first member of said binding pair coupled to a deposition nucleus and specifically binding said first member to a surface-immobilized second member of said pair and determining the electrical resistance of said surface, the method characterized in that after binding of the members on said surface an electrically conductive deposit is formed on said surface under conditions that allow said deposit to be formed specifically on said nucleus or deposit formed.

### FIELD OF THE INVENTION

The invention relates to the field of biology and the field of medicine. The invention in particular relates to means and methods suitable for use in an affinity sensor for detecting specific molecular binding events, as is particularly used in the molecular biological field, for example, in the medical diagnostics, in the biosensor technology or in the DNA-microarray technology, and application of the same.

### BACKGROUND OF THE INVENTION

Biosensors are solid phase measuring devices that are typically comprised of at least one biological receptor, a transducer and a subsequently connected electronic unit.

The receptor utilizes biologically active reagents such as, for example, antibodies for detecting a specific substance such as, for example, antigens. The transduction of detection events into detectable signals is performed by the transducer, for example, by electrochemical, optical, piezoelectric, or calorimetric methods. Thereby, the coupling of the detection events to the transducer can be carried out indirectly or directly. In the first case, the detection events modulate a process which is detected by the transducer. In the second case, the detection events themselves are recorded by the transducer. The transducer is connected to an electronic unit, for example, to a microprocessor followed by modules for signal detection and evaluation.

There are numerous application possibilities for such biosensors operating on the basis of molecular detection. These are, among others, in the field of detection and concentration analysis of biomolecules, the kinetic and equilibrium analysis of biochemical reactions, the control of fermentation processes, the evaluation of receptor-cell-interactions, the clinical analysis, and the cell detection.

The detection of the presence of bioactive molecules will be performed in the case of nucleic acids, for example, by hybridization with specific and marked nucleic acid probes. The marking of the probes is achieved by enzymatic inclusion of nucleotides that carry radioisotopes such as, for example, tritium, sulphur-35 or phosphorus-32, non-radioactive molecules such as, for example, digoxigenin or biotin and non-radioactive fluorescent molecules, respectively, such as, for example, fluorescein isothiocyanate or 7-amino-4-methylcumarin-3-acetate or metallic particles such as, for example, gold (Nicholl, D. S. T., 1995: Genetische Methoden, Spektrum Akademischer Verlag Heidelberg, p. 24-27).

In the case of antigens, such as peptides or proteins, the detection of the presence of bioactive molecules is achieved by specific and marked antibodies. The marking of the antibodies is performed by coupling of radioisotopes such as, for example, iodine-125 or tritium, to tyrosine-residuals and histidine-residuals, respectively, by non-radioactive enzymes, for example, alkaline phosphatase or peroxidase, whereby the enzymatic activity is measured, for example, by the conversion of a colourless product into a coloured one, by non-radioactive enzymes, for example, haematin which effects the chemiluminescent reaction of hydrogen peroxide and luminol, by non-radioactive enzymes, for example, luciferase which effects bio-luminescence by means of phosphorized luciferin, or by metallic particles such as, for example, gold (Liddell, E. and Weeks, L, 1996; Antikörpertechniken, Spektrum Akademischer Verlag Heidelberg, p. 87-107).

The signals from the various marker-molecules used will be evaluated by radio-chemical or electrochemical methods, by optical, by electric, piezoelectric, or calorimetric methods for indicating molecular detection events. Thereby, the size of the marker-molecules which emit single signals will lie in the nanometer range.

The optical and electrochemical methods for representing molecular binding events are the currently most utilized ones.

The problem of the various optical methods is, that the sensitivity and the spatial resolution of the signals emitted by the individual markermolecules is too low for many applications, that the binding between two links of a specific molecular binding pair cannot be detected, and that the signals are very often superimposed by an unspecific background. These problems of the image-generating methods can only be eliminated for a part by an experimental amplification of the signal or by a computer aided statistical image analysing method.

The technical limits of the current automation of the image analysing on the basis of the chip technology lies in the read-out of the various microarray spots. Most of the available technologies are based on the detection of the fluorescence marked binding pairs, which are held in a specific manner to the surface of the chip, whereby the fluorescence detection is performed by an optical read-out of the reactive centres of the microarrays. The application of fluorescent or chemiluminescent samples is thereby utilized just as in the conventional methods described hereinbefore and is combined with the CCD-imaging (Eggers, M. et al., 1996: Professional Program Proceedings, in Electro '96. IEEE, New York, N.Y., USA, 364 pp.; Heller, M. J., 1996: IEE Engineering-in-Medicine-and-Biology-Magazine 15: 100-104), whereby also here the mentioned problems of the conventional image analysing occur and a binding between two links of a specific molecular binding pair cannot be detected.

The detection of the presence of bioactive molecules can, also be obtained by an electrochemical approach by various methods, apart from the commonly used optical methods.

The measurement of redox potential variations in biomolecules is a well-known possibility, which is accompanied by specific binding events, for example, on enzymes. Thereby, the redox potential variations are measured by way of a single electrode, which is provided with biomolecules, and a reference electrode (Heller, A., 1992: Electrical connection of enzyme redox centres to electrodes, J. Phys. Chem. 96: 3579-3587).

The disadvantage of this method lies in the fact that only one single electronic event occurs for one bio-molecular binding event, whereby the variation of the redox state, which is effected, lasts only for a short time, so that the detection of each individual binding event had to take place flash-like. This is not possible. The signal obtained is only cumulative so that rare binding events cannot be detected by this technology.

A further possibility for detecting the presence of bioactive molecules in an electrical way is to use biosensors in the form of special measuring electrodes. Such special measuring electrodes generally are comprised of a (strept)avidin-coated electrode, whereby the (strept)avidin has the property to specifically bind biotin molecules. In this way it is possible to detect peptides, oligonucleotides, oligosaccharides and polysaccharides as well as lipids which are marked with biotin or biotin-derivatives, respectively to couple these as ligands to the (strept)avidin-layer. In the latter case, the biotin molecules are the coupling elements. Generally, these biosensors allow to detect antibody/antigen binding pairs, antibody/partial antigen binding pairs, saccharide/lectin binding pairs, protein/nucleic acid binding pairs, and nucleic acid/nucleic acid binding pairs. The detection of the biochemical events occurring at the special measuring electrode takes place in a similar way to that of the before described technology based on redox systems, namely, by measuring the potential variations across a single electrode compared to a reference electrode (Davis, et al., 1995: Elements of biosensor construction; Enzyme Microb. Technol. 17: 130-1035).

A substantial disadvantage of this conventional biosensor technology is the inherent low sensitivity of the measurements attained across the measuring electrodes that cannot be eliminated in that the ligands in an infinitely great density are bound to the measuring electrode, for example, by use of a dextran layer. Due to the additional deposition of, for example, a dextran layer and due to the spatial arrangement of the ligands, the concentration of ligands on the electrodes is indeed raised up to the sixfold compared to a ligand single layer, but a detection of rare binding events or even of a binding between two elements of a special molecular binding pair is not possible.

Further known possibilities are:

the anchoring of specific antibodies on a semiconductor gate of a field-effect transistor, whereby a variation in the charge distribution and, hence, in the circuit of the field-effect transistor is obtained by the selective binding of antigens to the special antibody layer;

the immobilizing of special antibodies on the surface of an optical fiber, whereby measurable optical phenomena such as, for example, interfering waves and surface plasmons appear due to the selective binding of antigens to special antibody layers at the site of intersection between the fiber optics and the liquid;

as well as the method of surface plasmon resonance, in which, at a definite angle of incidence of the light, the refractive index of a medium is, due to the selective coupling of antigens, measurably varied at a metal-coated glass body which is provided with specific antibodies (Liddell. E. and Weeks, L., 1996: Antikörpertechniken, Spektrum Akademischer Verlag Heidelberg, p. 156-158).

The disadvantage of these methods is that rare binding events cannot be detected by these technologies.

At present there are only a few methods available which allow a rapid detection of bindings between molecules at low concentrations or even with single molecule pairs (Lemieux, Bertrand et al., “Overview of DNA chip technology.” Molecular Breeding 4: 277-289, 1988), though the biochemical process of the binding pair formation with biosensors, for example, the hybridization of two nucleotide strands or the binding of antibodies to antigens itself runs very quickly, that is, within the range of seconds; biochips can be provided with binding molecules, for example, with specific oligonucleotides (U.S. Pat. No. 5,445,934) or specific proteins (U.S. Pat. No. 5,077,210) so that a chip technology will be possible (Osborne, J. C., 1994: Genosensors. Conference Record of WESCON/94.

Idea/Microelectronics. IEEE, New York, N.Y., USA: 434 pp.; Eggers, M. D. et al., 1993: Genosensors, microfabricated devices for automated DNA sequence analysis. Proc. SPIE-Int. Soc. Opt. Eng. 1998), by which the presence of definite biomolecules can be detected within a few minutes, for example, the presence of genes by use of specific oligonucleotide probes or antigens by use of specific antibodies, and by which great prospects are indicated in the field of biology or medicine, particularly as concerns genetic investigations (Chee, M. et al. 1996: Accessing genetic information with high-density DNA arrays. Science 274: 610614).

An alternative approach as concerns the detection of binding events between nucleic acid binding-pairs has recently been given by the utilization of the dielectric relaxation frequencies of the DNA to distinguish between hybridized and non-hybridized samples (Beattie et al. 1993. Clin. Chem. 39: 719-722). The detection of the differences in frequencies, however, requires equipment which still is very expensive and which, moreover, still is far from being utilized as a matter of routine.

Furthermore, there is known another way to electronically distinguish hybridized samples from non-hybridized ones, which consists in determining the speed of the electron movements along the DNA strands (U.S. Pat. No. 5,780,234). This determination is based on the fact th the arrangement of the pi-electron orbits in the double-stranded DNA causes the electrons to move faster in double-stranded DNA, that is, in hybridized DNA, than in single-stranded DNA (Lipkin et al., 1995: Identifying DNA by the speed of electrons. Science News 147, 117 pp.). To allow for a determination of these electron movements, the target has to be positioned exactly between two molecules. One of these molecules has to be chemically modified in such a way that it acts as an electron donor and the other one such, that it acts as an electron acceptor, so that there is a flow of electrons measurable via electrodes. This expensive method has the disadvantage that it limits its application to the detection of single-stranded nucleic acids fragments of a defined length and that it is not suited for further biomolecules.

Furthermore, one of the methods for an electrical detection of particles is known from Bezyadin, A., Dekker, C., and Schmid, G., 1997: “Electrostatic trapping single conducting nanoparticles between nanoelectrodes.” in Applied Physics Letters 71: 1273-.1275, in which nanoparticles are captured in a gap formed by electrodes in that a voltage is applied across the electrodes and the capturing of the particles is detected by way of the flow of the current. In contrast to the binding events of biomolecule pairs there is no specific biochemical binding of the nanoparticles, but the particle is bound to the electrode gap by the electric field.

There is also known from a work by Braun E., Eichen, Y,, Sivan, U., and Ben-Yoseph, G., 1998:“DNA templated assembly and electrode attachment of a conducting silver wire.” in Nature 391: 775-778, that DNA molecules can be held between two micro-structurized electrodes and these molecules only exhibited an electric conductivity after having been silver coated, whereby this conductivity has nothing to do with specific biochemical binding events of biomolecule pairs.

Alivisatos, A. P., Johnson, K. P., Peng, X., Wilson, T. E., Loweth, C. J., Bruchez Jr. M., P. and Schulz, P., G., 1996:“Organization of nanocrystal molecules using DNA” in Nature 382: 609-611, generated complexes from short single-stringed DNA-molecules and their complementary single-stringed DNA-molecules marked with gold particles in solution and deposited these on a TEM-grid with a carbon film for a characterization by electron microscope. An electric characterization, however, of the molecule pair binding did not take place.

Another method for detecting specific molecular binding events, is by way of an affinity sensor comprising a base on which electrodes are disposed in a spaced apart relation capturing a range that is provided with immobilized specified binding partners, which specifically couple complementarily associated binding partners, whereby said binding partners carry electrically conductive particles, so that there can be formed an electrically conductive contact between the electrodes and in this way the variation of the electrical resistance is detectable, when there is a potential applied across the electrodes, as well as the presence of single or a plurality of complementarily associated binding partners, carrying electrically conductive particles.

Using this method a variety of detection methods can be carried out for both single molecule as multi-molecule detection. A problem associated with the above-mentioned method is that the electrically conductive particles can comprise a charge that leads to repulsion of particles and thus of the associated molecules thereto. Moreover, also the molecules associated with the electrical conductive particle can comprise a charge thereby repelling similar molecules and thereby also repelling the electrical conductive particles associated with these molecules. For instance, nucleic acid in aqueous solution typically contains a charge thereby at least in part repelling other nucleic acid in that solution. Therefore, the electrical conductive particles associated with one member of a pair of complementarily associated binding partners tend to repel each other due to electrostatic interaction in the case that said member comprises a charge.

Repulsion of particles/labels and/or the associated molecules has the effect that once a binding event has taken place, it is more difficult, though not impossible, for additional binding events to take place in the immediate vicinity of the first binding event. This effect reduces the sensitivity and ease of work of a method for electrical detection of binding events, because this detection requires an electrical contact between the electrodes. At least part of the repulsion problem is counteracted by adopting a method which uses the so-called tunneling effect that allows a current to run between two physically separated nuclei, or conductive particles, that are bound to the surface of an electronic detection chip in close proximity of each other. However, the tunneling effect is limited to particles that are in close proximity. The effect rapidly decreases with increasing distance between particles. Therefore, very low concentrations of a member comprising an associated conductive particle are still difficult to detect when bound to its specific binding partner on a surface of an electronic detection chip.

### SUMMARY OF THE INVENTION

To flyer simplify methods for electrical detection of molecular binding events the invention in one aspect provides the electrical connection of two bound particles, or bound particle and an electrode, by depositing an electrically conductive deposit on bound particles/labels, deposit formed electrode or combination thereof. By the term bound particles herein is meant a particle or other label associated with a bound member of a pair of complementarily associated binding partners. The bound particle is in connection to the formation of a deposit thereon and is further referred to as a deposition nucleus. The deposition nucleus may be electrically conductive itself or not. The deposit as formed in a method of the present invention bridges the physical distance between two bound particles/labels, deposit formed, electrode or combination thereof, thereby creating either enhanced possibilities for the tunneling effects to occur when the particles are still essentially separated, or an essentially completely conductive path when the particles with thereon the deposit are essentially in contact with each other. In either case, the deposition of the invention will reduce the electrical resistance of the surface on which the particles/labels are bound. The deposition nucleus bound to the first member of a binding pair is not necessarily in contact with the surface, when binding between the first member and the second surface-immobilized member occurs. Therefore, the deposit may be deposited only and selectively on the deposition nucleus and not on the surface to which the second member is attached. This can be achieved for instance, by treating the surface such that deposition of deposit does not occur thereon, for example by silinization of the surface or providing thereto a substance such as poly(dimethylsiloxane). In such ways, deposition nuclei can be ‘grown’ into conductive particles by deposition of electrically conductive layer thereon. The treatment of providing a deposition as described herein does therefore does not necessarily result in a continuous layer or electron path between the electrodes on the surface, but can also result in a discontinuous electron path so that electrons tunnel from particle to particle between the two electrodes. An electrical measurement on such a discontinuous path on a surface for detection of binding between two members of a binding pair yield decreased resistivity across said surface.

In one aspect the invention therefore provides a method for detecting binding of members of a specific binding pair comprising providing a first member of said binding pair coupled to a deposition nucleus and specifically binding said first member to a surface-immobilized second member of said pair and determining the electrical resistance of said surface, the method characterized in that after binding of the members on said surface an electrically conductive deposit is formed on said surface under conditions that allow said deposit to be formed specifically on said nucleus or deposit formed. By virtue of the possibility of deposit forming on deposit, the layer of deposit is capable of increasing in thickness.

The invention further provides a sensor comprising electrodes in contact with a surface comprising a binding pair wherein a member of said binding pair comprises a deposition nucleus and wherein said deposition nucleus comprises an electrically conductive deposit. The sensor in this aspect can directly or after further processing be used to determine the electrical resistance of said surface.

A specific binding pair comprises at least two molecules capable of specifically binding to each other. Preferred binding pairs comprise complementary nucleic acids strands, or derivatives and/or analogues thereof such as PNA and proteinaceous molecules capable of specifically binding to each other, such as but not limited to, receptor ligand interactions or antibody-antigen interactions. Binding pairs can also comprise of combinations of nucleic acid and proteinaceous molecules or carbohydrates and any other combination of molecule types as long as the interaction between the binding partners is specific. Each binding partner may comprise one or more molecules, for instance in the form of a complex. For instance, the surface immobilized member of the binding pair may comprise an antibody bound to an antigen, wherein the antigen is capable of binding to said member of said binding pair comprising said deposition nucleus. Specific binding of the pair means that binding between the partners of the pair has a higher affinity and/or lower dissociation constant than interactions of each of the partners with the large majority of other molecules. It is within the scope of the present invention that one or both partners of the binding pair can specifically bind to a limited number of other molecules. Nucleic acid hybridizations, for instance, are possible between two completely complementary nucleic acid strands, but also between strands that comprise one or more mismatches depending on the specific circumstances such as the sequence length and the hybridization conditions. Similarly, a receptor may be able to bind several different ligands, or the derivatives of the same ligand, or vice versa. The definition of specific binding is not meant to exclude these or similar interactions.

An electrically conductive deposit is formed on said surface under conditions that allow said deposit to be formed specifically on said nucleus or deposit formed. Deposit should not form on the surface-immobilized member in the absence of a deposition nucleus. Electrodes on the surface of the sensor may function as deposition nucleus. However, the efficiency of deposition on electrodes should preferably not drastically exceed the efficiency of deposition on deposition nuclei associated with a member of a binding pair. The catalytic properties of the deposition nucleus determine for a large part the efficiency of deposition of the electrically conductive deposit on the deposition nucleus.

The efficiency of deposition is also dependent on the conditions used to deposit the electrically conductive deposit. A person skilled in the art is well capable of selecting an appropriate deposition nucleus and deposition conditions. A person skilled in the art may use the conditions used in the present invention or use conditions in the art (Bezryzin et al, 1997, Appl. Phys. lett. 71:1273-1275; Reed et al, 1997, Science 278:252-254; Braun et al, 1998, Nature 391:775-778; Velev et al, 1999, Langmuir 15:3693-3698; Brown et al, 2000, Chem. Mater. 12:314-323; Musick et al, 1997, Chem. Mater, 9:1499-1501; Park et al, 2000, Angew. Chem. Int. Ed., 39:3845-3848).

Electrical conductivity is herein defined as the transfer of an electric current through a solid or liquid, preferably through an electrically conductive particle and/or deposit, from one electrode to another electrode, and between which electrodes is (generated) a potential difference or which electrodes are connected to either end of a voltage circuit. Conductivity is inversely proportional to the resistivity.

Suitable deposition nuclei comprise an electrically conductive material comprising a metal, such as for instance pyrite, stannous chloride or palladium chloride. Also suitable are non-metal deposition nuclei like graphite or organic polymers. In fact the deposition nucleus itself is not necessarily electrically conductive, but it must be catalytically active to induce conductive (metal) deposition from solution. Therefore, also suitable deposition nuclei comprise nuclei with surfaces that are catalytically active with regard to deposition of conductive deposits from solution.

In a preferred embodiment said deposition nucleus comprises gold, silver, iron, copper, nickel or mixtures comprising of one or more of said metals. Preferred deposition nuclei may also comprise non-metal substances such as polymer beads with catalytically active substances on their surface. In an especially preferred embodiment said deposition nucleus comprises gold. Gold and particularly gold colloids are particularly suited because its surface is catalytically active as described above.

The electrically conductive deposit formed is typically chosen on the basis of suitable conditions for depositing it on the deposition nucleus. Thus the choice of nucleus and the choice of deposition conditions are connected to each other based on the relation on catalytic deposition activity of the nucleus towards the depositing compound. In a preferred embodiment said deposit comprises an electrically conductive metal, preferably silver, gold, copper, nickel, lead, mercury, and the like. In a particularly preferred embodiment said deposit comprises silver. Silver may be deposited under suitable conditions from aqueous solutions comprising silver ions and a redox-active substance that transfers electrons on to the silver ions.

The deposition nucleus comprises a particle comprising the material on which said deposit is to be formed. Suitable sizes for the particles range from 0.1 nm to 5 μm. Preferably, the size of the particles ranges from 0.8 nm to 250 nm. The latter size range allows easy coupling to a molecule selected to be a member of a binding pair and easy production of colloids (Hayat, 1970, Hayat (ed) Van Nostrand Reinhold, New York, N.Y. USA).

Since gold nanoparticles exhibit surface-adsorbed ion layers, such particles will repel each other, which leads to a stabilizing effect. So any addition of counter-ions should be well balanced since too many ions will result in screening effects and thereby result in insufficient repelling and as a result thereof to aggregation of the gold particles. Therefore when using gold or other charged particles in a method of the present invention in combination with e.g. nucleic acid hybridization—which hybridization conditions require salts—the ionic strength should be optimized in order to avoid aggregation. It is within the scope of the invention that more than one member may be associated with one deposition nucleus. However, the invention also works if said deposition member contains one member of said binding pair.

The binding pair may comprise any type of molecules. It is not required that both members of the binding pair are known or even identified prior to using the invention. A surface-immobilized second member may, for instance, be used to determine whether a sample comprises a first member capable of specifically binding to said second member. In a preferred embodiment said binding pair comprises biological molecules. In a preferred embodiment said binding pair comprises a proteinaceous molecule. A proteinaceous molecule comprises at least two amino acids or derivative thereof lined though a peptide bond. Preferably, said proteinaceous molecule comprises at least 10 and preferably at least 15 amino acids or derivatives thereof linked through peptide bonds. A derivative of an amino acid is an amino acid, occurring in nature or artificially synthesized, wherein at least one functional group of said amino acid is altered prior to but preferably after peptidic linking.

In another preferred embodiment said binding pair comprises nucleic acid or a functional derivative and/or analogue thereof. A derivative or analogue of nucleic acid comprises the same hybridization characteristics as nucleic acid, in kind not necessarily in amount. A suitable derivative of nucleic acid is nucleic acid that is chemically modified, for instance through the addition or interchanging of functional groups on the sugar moiety. A suitable analogue of nucleic acid comprises PNA.

The invention also provides a method for quantifying a member of a specific binding pair in a sample comprising

linking the member in said sample with a deposition nucleus,

contacting a surface comprising an immobilized second member of said pair with said sample under conditions allowing specific binding of said pair,

subsequently performing an enhancement step on said surface by depositing an electrically conductive deposit on said nucleus and/or deposit formed, and

determining the electrical resistance of said surface and optionally comparing the electrical resistance of said surface with a reference. Quantification can be achieved in various ways, for instance by measuring the assessing the amount of electrical resistance of said surface. Preferably however, quantification is performed by comparing the electrical resistance with a reference. The comparison may be with a reference electrical resistance or range thereof measured for a standard concentration or standard range of concentrations of said first member associated with similar immobilization surfaces. The comparison may however, also be with a standard amount or standard range amounts of deposition nuclei associated with similar immobilization surfaces. In a preferred embodiment said reference comprises a second surface on which said enhancement step was performed essentially in parallel and wherein said second surface comprises a known amount of said deposition nucleus. In a further preferred embodiment said second surface comprises a known amount of first member comprising said deposition nucleus and wherein said first member was specifically bound to said immobilized second member essentially in parallel to said sample. Preferably said reference comprises a set of surfaces wherein each surface comprises a different known amount of first member comprising said deposition nucleus, and on which said enhancement step was performed essentially in parallel with said sample.

One enhancement step can be performed to perform the invention, particularly with a relatively large amount of deposition nucleus is immobilized on/the immobilization surface. Preferably, however, at least one further enhancement step is performed. This will facilitate the detection of smaller amounts of deposition nucleus associated with the immobilization surface. Between each enhancement step the electrical resistance of the surface may be measured. In a preferred embodiment said enhancement step is repeated until the electrical resistance in said reference indicates the detection of binding of a deposition nucleus to said reference surface. Preferably, said enhancement step a repeated until the an electrical resistance indicative for binding of a deposition nucleus on the surface that was exposed to said sample in a method of the invention is detected.

### DETAILED DESCRIPTION OF THE INVENTION

The invention will be explained hereinafter in more detail by virtue of schematical embodiments under reference to the drawings. There is shown in:

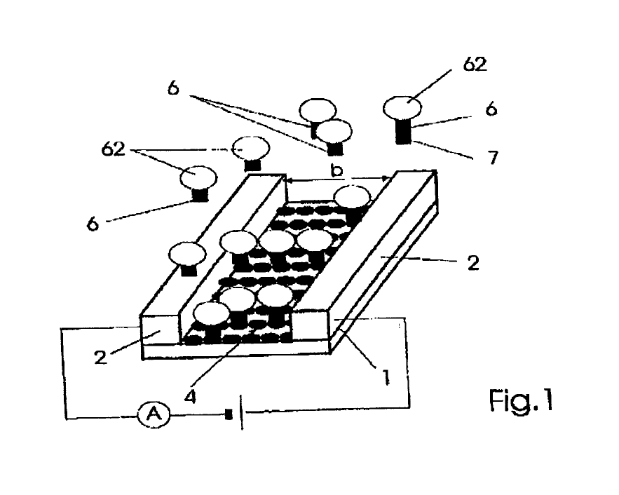

FIG. 1 an affinity sensor for detecting specific molecular binding events;

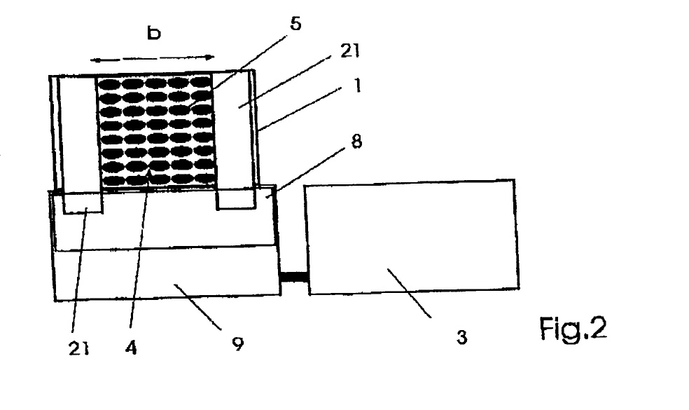

FIG. 2 a schematical representation of the affinity sensor for detecting specific molecular binding events,

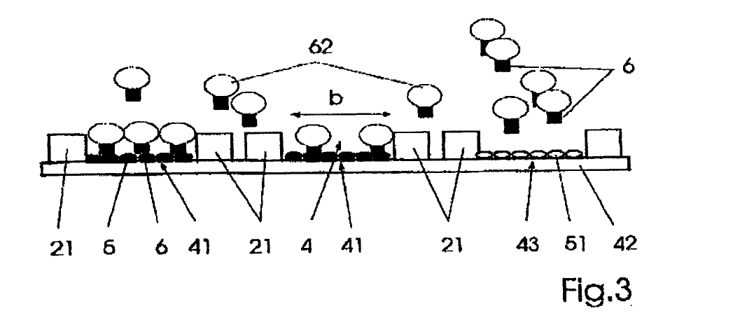

FIG. 3 a cross-sectional view of an embodiment of the affinity sensor for detecting specific molecular binding events;

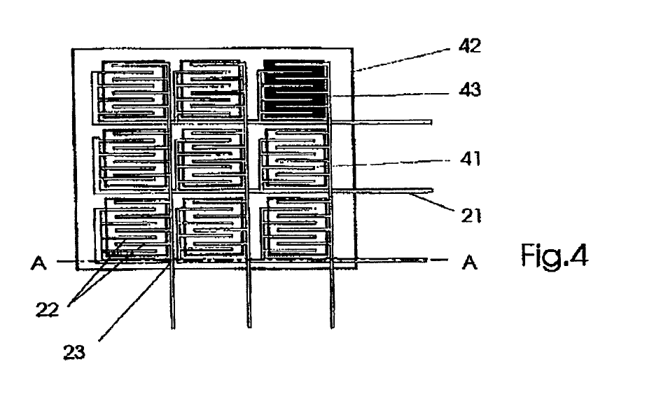

FIG. 4 a plan view of an embodiment of the affinity sensor in the form of an affinity chip;

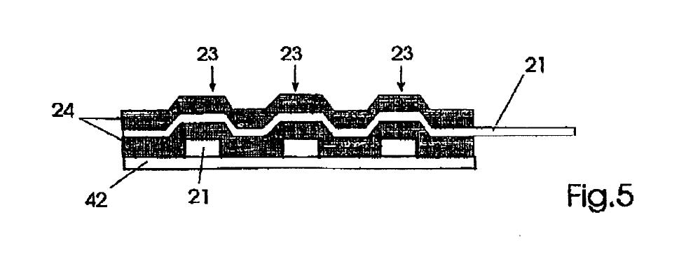

FIG. 5 a sectional view along the plane A-A of the affinity chip represented in FIG. 4;

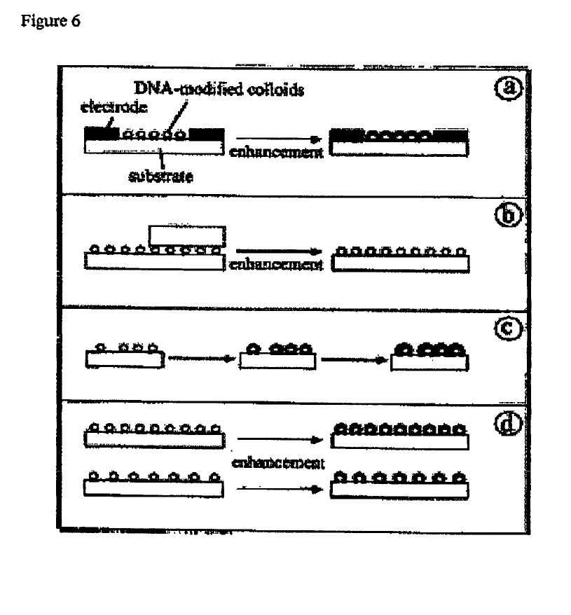

FIG. 6 a schematic representation of the general principle of enhancement-induced electrical conductivity, wherein gold colloids situated in an electrode gap are enhanced using elecroless silver deposition which at first leads to reduction of the distance between the particles on the surface and which ultimately leads to a conductive path between the electrodes;

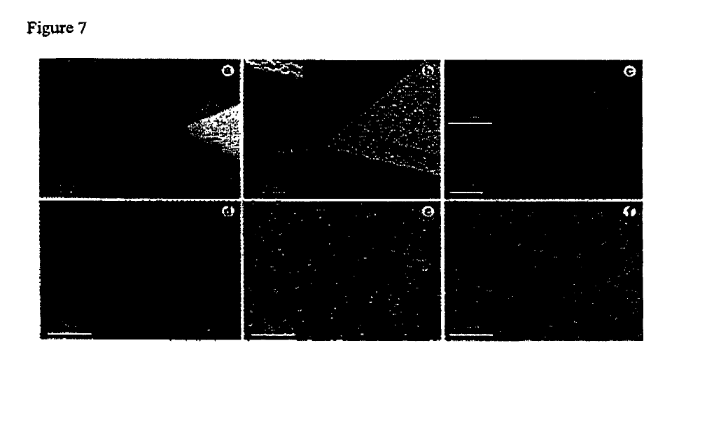

FIG. 7 a series of scanning electron micrographs (SEM, top) and scanning force microscopy (SFM, bottom) of silver enhancement of colloidal gold particles bound by DNA-DNA interaction (hybridization) in an electrode gap; wherein in FIG. 7a is show a sample before enhancement with the electrode structure (gold) visible to the right and a particle-covered substrate composed of silicon oxide (in this figure, the 30 nm particles are hardly visible, even in a zoom (inset)); wherein in FIG. 7b a sample after silver enhancement is shown, wherein the inset shows a zoom of the region adjacent to the electrode; wherein in FIG. 7c is shown an experiment wherein the right part of the surface was covered during the enhancement, so that the silver deposition occurred only in the left part (top-SEM, bottom-SFM). An image such as FIG. 7c allows a direct characterization of the enhancement efficiency by comparing enhanced (left) and original (right) particles; wherein in FIG. 7d (DNA-modified particles no enhancement) 7e (first enhancement) and 7f (second enhancement) is shown the effect of repeated enhancement as observed by SFM;

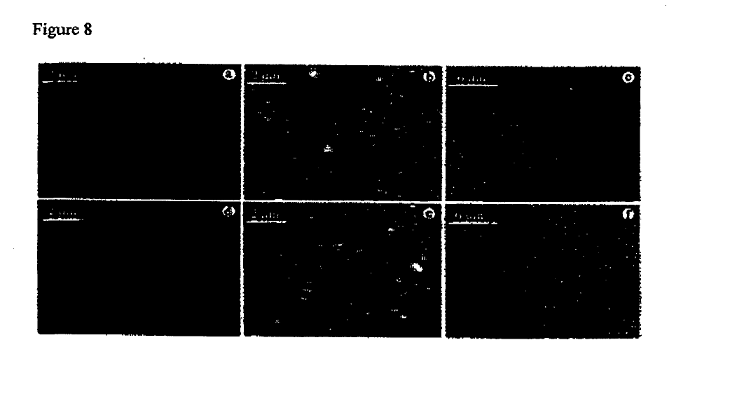

FIG. 8 the parallel enhancement of samples with different surface densities of gold particles. DNA-modified particles were adsorbed onto silicon oxide surfaces. The upper row shows the results with the high-density sample; images of the lower density sample are in the lower row. Samples with high/low density were SFM-imaged before (a/d) and after (b/e) enhancement. SEM-images of the enhanced surfaces are also shown (c/f).

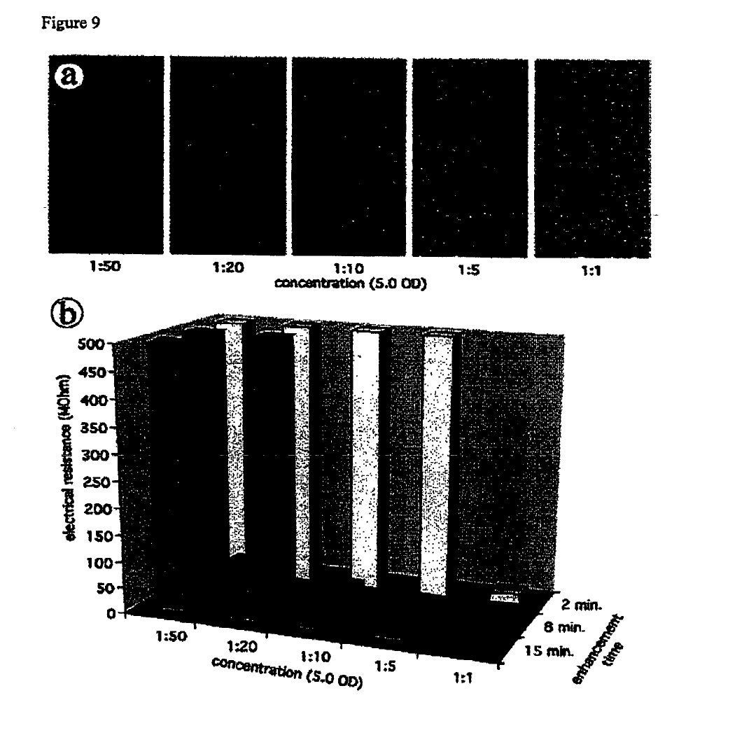

FIG. 9 the electrical classification of a concentration series of DNA-nanoparticle solutions. Five different solutions with concentrations between 5.0 OD and 0.025 OD were hybridized to DNA-substrates as explained in the Example described below, resulting in different surface densities of particles (a). These samples were enhanced for different times, and the resistance was measured (b). The applied current was limited (to avoid destruction of the electrodes), resulting in a cut-off value of 500 MOhm.

The affinity sensor for detecting specific molecular binding events shown in FIGS. 1 and 2, is comprised of a carrier substrate 1 which is provided with electrodes 2 enclosing a range 4 that is provided with immobilized specific binding partners 5. Thereby the range 4 represents a discontinuity in an electric circuit that includes an amplifier circuit 8, which can be part of a microchip 9, as well as a measuring and evaluating unit 3, whereby in the present example the electrodes 2, which limit the range 4, are associated to the electric circuit and define a minimum width b of the range 4. The specific binding partners 5 are capable of coupling complementarily associated binding partners 6 specifically and directly or via further specific binding molecules 7, whereby the complementarily associated binding partners 6 including deposition nucleus 62 are directly coupled or via binding molecules. The range 4 is, by the arrangement of the electrodes 2, so dimensioned in its width and effective height tat the coupling of the immobilized specific binding partners 5 to the complementarily associated binding partner 6 which carry the deposition nucleus 62 or, via further specific binding molecules 7, with the complementarily associated binding partners 6 which carry the deposition nucleus 62. Provided that the specific binding partners 5 are realized by the molecules of a nucleic acid probe species, the complementarily associated binding partners 6, which carry the deposition nucleus 62, by nucleic acids and the deposition nucleus 62 by nanoparticles of a size of 20 nm, then the minimum width b of the range 4 is 25 nm and its effective height 20 nm.

In the event that the deposition nucleus comprises an electrically conductive particle, the coupling of the specific binding partners 5 in the range 4 to the complementarily associated binding partners 6 carrying the deposition nucleus 62 effects, when there is applied a voltage across the electrodes 2 (refer to FIG. 1), the motion of the electrons via the electron transport barrier in such a way that the deposition nucleus 62 bridge the range 4 so that the electrons tunnel from electrically conductive particle 62 to electrically conductive particle 62 and to the electrodes 2, as a result thereof a permanent variation of the electric resistance across the range 4 between the electrodes 2 can be measured by aid of the post-connected amplifier circuit 8 in combination with the measuring and evaluating unit 3.

The measurements can also be performed in a humid environment, in particular by aid of a gel layer, instead of measuring in a dry state.

In order to enhance the electric conductivity of the range 4 between the electrodes 2, which is achieved by way of the complementarily associated binding partners 6 in cooperation with an electrically conductive deposition nucleus 62, already known electron-transfer-mediators or effective diffusing electron donors and electron acceptors can be used, such as water soluble ferrocene/ferricinium, hydroquinone/quinone, reducible and oxidisable components from organic salts, cobaltocenes, hexacyanides and octacyanides of molybdenum, tungsten, and iron, respectively, macrocycles and chelating ligands from the transition metals such as cobalt, ruthenium, and nickel, including Co(ethylenediamine)3- and Ru(ethylenediamine)3- and trisbipyridyl and hexamine-complexes from transition metals such as Co, Ru, Fe, and/respectively, organic molecules such as 4-4′-bipyridines and 4mercaptopyridines, which are free in solution or present in a gel deposited on the carrier substrate 1 or in a polymer deposited on the carrier substrate 1. When a known gel-based matrix immobilization utilizes nucleic acids as specific binding partners 5 then, due to the three-dimensional structure of the polymer, it exhibits the advantage that a greater number of capturing ligands is immobilized on the small surface section of the range 4. By using a highly porous hydro-gel, the hybridization rate, for example, of the nucleic acids which are the specific binding partners 5 and the complementarily associated binding partners 6, which carry the deposition nucleus 62, is increased and lies within ranges as they are known for nucleic acids in solution.

The affinity sensor shown in FIGS. 3 and 4, which is in the form of a affinity chip, is characterized in that the electrodes 2 are designed as micro-electrodes 21, which are arranged in two pairs each, capturing a respective affinity area 41. Thus, a matrix of affinity areas 41 results, which is adapted to simultaneously and electrically detect in the different interspaces 4 a plurality of various couplings.

Thereby, the individual affinity areas 41 are designed in an interdigital electrode structure arranged upon a chip surface 42. The chip surface 42 consists of silicon or glass upon which, for example, a dielectric oxide layer is provided. Due to the digitally branched microelectrodes 21, which, for example, can be manufactured to yield the shape of comb-like electrodes 22, the ranges 4 on the affinity area 41 can be defined to have a length within a range of 20 gm. The microelectrodes 21 are spaced apart and electrically separated from each other by an interposed insulating layer 24, as shown in FIG. 5, which is provided at the intersections 23 of the micro-electrodes 21. Thereby and provided that the specific binding partners 5 are realized by the molecules of a nucleic acid probe species, the complementarily associated binding partners 6, which carry the deposition nucleus 62, are nucleic acids and the deposition nucleus 62 are nanoparticles of a size of 20 nm, then the ranges 4 have an effective height of 100 nm and a width of 200 nm. Consequently, at least one coupling, which establishes a contact between the microelectrodes 21, is achieved between the immobilized specific binding partners 5 and the complementarily associated binding partners 6 that carry tee deposition nucleus 62. In this example, the immobilized specific binding partners 5 are capturing ligands in the form of nucleic acid probes and the complementarily associated binding partners 6, which carry the deposition nucleus 62, are target molecules in the form of nucleic acids. The oligonucleotide probes immobilized as specific binding partners 5 are bound to the silanized carrier substrate 1 via an amino group, whereby a probe density in an order of size of 10,000 molecules per μm² is attained in this example. The complementarily associated binding partners 6 are oligonucleotides in this example, which are marked with gold particles, the hybridization conditions depending on the respectively used probes.

Alternatively, the affinity areas 41 can be provided with various immobilized specific binding partners 5 in sectors, which are respectively separated from each other.

Affinity areas 41 with immobilized specific binding partners 5 and reference areas 43 with immobilized inactive binding partners 51 are provided on affinity chips, represented in FIGS. 3 and 4, so that the measurement of the electric resistance between the micro-electrodes 21 is carried out as a reference measurement of the electric resistance between an affinity area 41 and a reference area 43, whereby the micro-electrodes 21 can be designed as comb-type electrodes 22. Thereby the immobilized specific binding partners 5 and the immobilized inactive binding partners 51 can be of a thickness which, when covering the electrodes 21, permits the tunnel effect, rendering the manufacture of the chips technologically more easily.

Since the reference area 43 is free from immobilized specific binding partners 5, due to the occupation by inactive binding partners 51, this space between the two micro-electrodes 21, insulated from each other, represents an electrical barrier so that there does not take place a measurable electron transfer between them. Also, since the reference area 43 is free from immobilized specific binding partners 5, no or at least substantially less electrically conductive deposit is formed on the surface of the reference area 43 under conditions that allow said deposit to be formed in a method of the invention.

The affinity area 41, which in contrast thereto carries immobilized specific binding partners 5, binds via the latter and through the coupling event the complementarily associated electrical binding partners 6, which carry the deposition nucleus 62, so that as a result thereof, by the particles 62, the space of the affinity areas 41 between the micro-electrodes 21, which are designed as comb-type electrodes 22, is divided into a plurality of gaps of nanometer width. The nano-gaps formed by an electrically conductive deposition nucleus 62 result in that an electron transfer is possible between the two contact faces of the micro-electrodes 21 by virtue of the tunnel effect, so that the variation of the resistance can be detected via the amplifier circuit 8 by means of a measuring and evaluating unit 3, when there is a voltage applied across the microelectrodes 21. In the present example, the voltage applied lies in an order of size of less th one volt.

Alternatively to the measurement of the potential applied across the affinity 41 by an electrode system comprised of reference electrode, sample electrode and counter electrode, it is also possible to employ other methods of an electrical detection such as, for example, potentiometric and voltametric measurements.

Standard chemical linkers such as, for example, amino-modified ligands, are used to immobilize the specific binding partners 5 and the inactive binding partners 51, respectively, such as, for example, antibodies or nucleotide probes, so that the chemical linkers are bound to the silanized chip surface 42 and constitute the affinity areas 41 and the reference areas 43, respectively.

The marking of the complementarily associated binding partners 6 such as, for example, protein targets or the target nucleic acid, by means of deposition nucleus 62 is performed according to the known methods such as, for example, the final marking with marked oligonucleotides, by utilizing ligases.

In the following, the manufacturing of affinity sensors according to the present will be described in more detail. In a preferred embodiment the affinity sensor is comprised of a plurality of ranges 4 (also referred to as detection ranges), whereby each of which is captured by at least two electrodes 2. These detection ranges are provided with specific binding partners (capture molecules) 5 such as antibodies, fragments of antibodies or DNA-, RNA- or PNA-oligonucleotides, to which definite associated binding partners (target or detection molecules) 6 may bind in a specific manner. The specific binding partners 5 are defied as marked or non-marked molecules, which can be selected for being bound to the desired target molecule in the ranges 4 of the affinity sensor. To this end, not only conventional (bio)molecular binding pairs can be utilized as capturing molecules, as target molecules and as detection molecules, but also specific chemical binding pairs as known from the combinatorial chemistry, which can also be utilized as binding pairs within the frame of the invention. The formation of this described specific binding can be understood as a primary binding event. It is possible to carry out the detection of this primary binding in a one-step procedure or in a multi-step procedure, e.g. by intermittent binding of associated binding partners (target molecules) 7 to specific binding partners (capture molecules) 5, whereby the specific co-immobilization of the material, which transfers the electrons, for example, the gold particles 62, is carried out in the last step via the binding of definite associated binding partners (detection molecules) 6 to associated binding partners (target molecules) 7. This latter embodiment is known as a sandwich-type detection method as known in the art. In such an embodiment the specific binding partners (capture molecules) 5 are essentially complementary only to associated binding partners (target molecules) 7 and not to definite associated binding partners (detection molecules) 6, while at the same time definite associated binding partners (detection molecules) 6 are essentially complementary only to associated binding partners (target molecules) 7 and not to specific binding partners (capture molecules) 5. The binding between specific binding partners (capture molecules) 5 and associated binding partners (target molecules) 7 is then detected through co-immobilization of the definite associated binding partners (detection molecules) 6. This co-immobilization can be performed by specific kinds or unspecific kinds of molecular interaction, such as a hybridization of probes marked with gold onto the desired target molecule or by a direct marking of the target molecule with the properties of an electron transfer in such a way that this marking can be electronically detected. The mentioned co-immobilization is, in principle, separated from the primary binding event, but in dependence therefrom and can be performed simultaneously. Thus, the co-immobilization or attachment of material, which transfers electrons, to the designated surface of the affinity sensors can be taken as an indirect result of the primary binding. The detection of this co-immobilization is obtained by an electronic measurement of the variation of the electric conductivity across the measuring range, this variation of the electric conductivity being an indication of the presence of target molecules.

The primary binding or co-immobilization of electron-transferring material can be exploited to induce secondary depositions which are adapted to transport electrons. It lies within the scope of the present invention that the specific binding of target molecules can be detected by way of a multi-step process, which comprises at least one step by way of which electron-transferring material is deposited, this material effecting a reduction of the electric resistance across the measuring range. It is possible to use organic or inorganic substances or compounds for the electron conductive particles 62. This conductivity is used for detecting and marking of the desired target molecule, that is, for detecting the presence thereof.

The electron conductive particles 62 may also be prepared by a method of the invention is such a way that first a nucleus is provided onto which an electron-transferring material is deposited. As described above, this nucleus comprises a surface that is catalytically active with regard to deposition of conductive deposits from solution. The deposit as formed in a method of the present invention thereby enlarges the nucleus into an electron conductive particles 62, thus bridging the physical distance between two bound particles/labels thereby creating either enhanced possibilities for the tunneling effects to occur when the particles are still essentially separated, or an essentially completely conductive path when the particles with thereon the deposit are essentially in contact with each other. Thus the electrical resistance of the immobilization surface may be lowered through both continuous as well as dis-continuous deposit. The latter occurring through the so-called tunneling effect. In either case, the deposition of the invention will reduce the electrical resistance of the surface on which the particles/labels are bound.

### EXAMPLES

In the following and without limiting the present invention thereto there will be described several possibilities of preparation steps for manufacturing an affinity sensor according to the present invention.

A. To prepare the required electrodes, a silicon wafer having on one side an oxide layer of about 1 μm thickness is coated by sputtering with a bonding layer, for example, of 3 nm Ti, to said oxide layer and a gold layer of a thickness of 50-100 nm. To be able to provide for the electron gap width in the lower nanometer range, a multi-layer masking is utilized for the micro-structuring. To this end, a coating with a carbon (30 nm) is performed, followed by a coating with a metal combination (Ti and NiCr, respectively, of a thickness of 10 nm). Subsequently, an electron beam resist (150 nm) is deposited by spinning-on. The exposure is realized by a mix-match-technology, in the course of which the large-area electrodes 2 are generated by means of a shaped-electron-beam exposure device and the minute gaps between the electrodes 2 by means of a point-beam electron-beam exposure device. The structure is transferred to the metal layer by ion beam etching (IBE) and to the carbon layer by a reactive ion-etching (RIE). The transfer of the structure to the gold-layer and the bonding layer is carried out by way of an IBE-process as known in the art. Finally, the masking layer is removed in a RIE-process as known in the art at a simultaneous surface activation.

In the following, techniques will be described which are based on a silanization of the surface of the chips. Due to this silanization, the surfaces are activated for binding amino-modified oligonucleotides: Two different methods for the silanization and subsequent immobilization will be explained here. Of course, there are also other possibilities for surface activation and immobilization, apart from the silanization.

B1. Silanization by application of 3-aminopropyltrimethoxysilane APTES.

The pre-structured chips with gold electrodes, as described by example under A., are purged in an ultrasonic bath and, in sequence in concentrated nitric acid, in hydrogen peroxide solution (30%) and water, and subsequently dried for 5 minutes at 80° C. Then the chips will be incubated for 2 min. in a 1% silane solution in 95% acetone/water. After having been washed for ten times in acetone for 5 minutes each, the chips will be dried at 110° C. Then they will be incubated for 2 h in a 0.2%-phenylenediisothiocyanate solution in 10% pyridine/dimethylformamide and washed with methanol and acetone. Chips activated in this manner can be stored in a desiccator at 4° C. for a longer time.

Subsequently, the linkage of the amino-modified oligonucleotides is performed, to this purpose a drop of the oligonucleotide solution (2 mM in 100 mM sodium carbonate/sodium bicarbonate buffer) is deposited upon the chip. The parallel application of small drops of different oligonucleotides allows a parallelization, for example, by use of an embodiment of the affinity sensor according to FIG. 4. The deposition of the mentioned drops can be performed by means of micro-pipettes, spotters or other available techniques suited for the application of small amounts of samples. Then, the chips are incubated in a moisture chamber at 37° C. for about 1-2 h. After removal of the drops the chips will be washed with 1%-ammonia solution for onetime, and three-times with water. Then drying is carried out at ambient temperature.

B.2. A second possibility of silanization is carried out by application of 3-glycidoxypropyltrimethoxysilane (GOPS), to this end, as described under B1., the chips are purged and subsequently are treated in an ultrasonic bath, each for 12 min. with hexane, acetone and ethanol. Then tie chips are dried for 5 minutes at 80° C. The silanization is carried out with 1 mM GOPS in dry toluol at 80° C. for 6-8 h. The chips are thoroughly washed with ethyl acetate and are ready for immediate use.

Subsequently, the linkage of the amino-modified oligonucleotides is performed. To this purpose a drop of the oligonucleotide solution (5-50 μM in 0.1 M KOH) is deposited upon the chip and the chip is incubated in the moisture chamber at 37° C. for 6 h. Again a parallelization, as referred to under B.1. can be obtained due to the deposition of a plurality of drops with different oligonucleotides. Then the drops are allowed to dry, and then washing is carried out with water at 50° C. under continuous shaking, followed by drying at ambient temperature.

C. In this put of the specification there will be described the possibility of marking oligonucleotide probes with colloidal gold. To start with, there is required a preparation of the thiolated oligonucleotide, which is carried out as follows: the 3′-alkylthiol modified oligonucleotides are solid-phase bound to a dithiolcompound by the manufacturer to protect its functional group. By separation from the carrier material the functional group will be released and is then in the active state. The separation takes place in 50 mM DTT (dithiothreitol) in concentrated ammonium hydroxide at 55° C. for 16 h (original solution. 4-8 mg solid-phase bound oligonucleotide, 450 gl water, 50 gl 1M DTT, 50 gl cc ammonium hydroxide). After incubation the liquid phase is separated from the solid phase (Controlled Pored Glass, CPO) and desalinated by way of column chromatography. The oligonucleotides are then washed out in reaction buffers. The concentration of the single chromatography fractions is then detected by a spectrophotometer.

The reaction solution will be incubated at 55° C. for 16 h at 600 revolutions per minute in a thermomixer, and then centrifuged for 23 min. at an acceleration of about 16,000 m/s2. Fractions that are prepared in this manner can be stored for more than 4 weeks at −20° C.

The binding of the thiolated oligonucleotides to colloidal gold will be described by example in the following:

There are added to 5 ml gold solution (about 17 nM) 2.5 OD (260 nm) alkylthiololigonucleotides [OD (260 nm)=optical density at 260 nm], (final concentration 3.6 nM). Subsequently to a pre-incubation for 16 h at ambient temperature, incubation is carried out after a setting to 0.1 M NaCl/10 mM sodium phosphate buffer (pH 7.0) for 40 h at ambient temperature. Thereafter, again a centrifugation takes place for 25 min. at an acceleration of about 16,000 m/s². The resulting pellet is washed with 5 ml 0.1 M NaCl/10 mM sodium phosphate buffer (pH 7.0), followed by a further centrifugation for 25 min. at an acceleration of 16,000 m/s². The re-dispersion is carried out in 5 ml 0.3 M NaCl/10 mM sodium phosphate buffer (pH 7.0).

40 μl of the aqueous solution with colloidal gold particles (diameter of 30 nm in the example) obtained in the above described manner are placed in the range 4 between the electrodes 2. After drying, electric measurements, which have been described herein further up, show a linear current-voltage characteristic which is indicative of an ohmic behaviour of the aggregated gold colloids in the range under consideration. A current of 0.3 μA was measured at a voltage of about 0.3 volt applied across the electrodes 2.

The affinity sensor as, for example, disclosed in connection with FIGS. 3 and 4 and in form of the affinity chips, can find a variety of applications as, for example, in the molecular biology and in the medical diagnostics where specific bindings of bioactive molecules to their corresponding binding partners, for example, DNA, proteins, saccharides are to be determined.

Based on the electrical detection of specific molecular binding events, the affinity sensor allows to perform a bio-monitoring of, for example, molecules, viruses, bacteria, and cells in the most diverse samples, for example, in clinical samples, in samples of food and from the environment such as, for example, from clarification plants, whereby such monitoring is performed in a quick, sensitive and specific way.

In the following description of modes of the invention examples are given of means and methods for depositing an electrical conductive deposit on a deposition nucleus associated with an immobilization surface.

Materials and Methods Substrate Preparation

Electrode structures with dimensions in the 10-100 μm range were prepared from a silicon oxide substrate covered by 5 nm titanium and 100 nm gold using standard photolithographic processes. The substrates were activated by oxygen etching prior to use either for direct adsorption of the modified colloids or for covalent binding of amino-modified DNA

DNA-Modified Surfaces and Particle Binding

Amino-modified DNA, which serves as binding partner for the complementary DNA immobilized on the colloidal gold, was immobilized on the activated silicon oxide substrate using a silanization step as described under B2.

For the DNA-modification of colloidal gold (30 nm diameter, British Biocell), 3′-alkylthiolated oligonucleotides (BioTeZ, Berlin, Germany) were used as described previously (Möller R., Csaki A., Köhler, J. M., Fritzsche, W. Nucl. Ac. Res. 2000, 28, e91.). Two different complementary associated binding pairs of complementary DNA were used resulting in a double strand of 12 (FIG. 7) and 60 (FIG. 9) base pairs, respectively.

For specific binding, droplets of the DNA-modified gold nanoparticle solution were placed on a the surface of a DNA-modified chip (surface with immobilized DNA) and incubated in a covered Petri dish containing a small amount of water. The chips were incubated for 1 h at 44° C. and then cooled to room temperature. Afterward they were washed once with buffer (0.3 M NaCl/10 mM sodium phosphate, pH 7.0) and deionized water, and then air-dried. For nonspecific binding, droplets of the nanoparticle solution were applied to a freshly activated silicon oxide chip and incubated in a moisture-saturated atmosphere at room temperature for several hours, then washed with buffer and water, and then air-dried.

Because the enhancement efficiency and kinetics are independent of the kind of surface immobilization, both cases (specific and nonspecific particle binding) were comparable in terms of enhancement time and measured resistance.

Silver Enhancement

Before the enhancement, the chips with immobilized particles were thoroughly washed with deionized water. Then droplets of the enhancement solution (Silver Enhancing Kit, British Biocell) were applied to the chip and the chips were incubated at room temperature for 15-20 min. Better results were achieved when the smaller chips were placed directly in the enhancement solution in an Eppendorf cup, compared to the deposition of droplets onto the chips.

Electrical and Microscopical Characterization

A Keithley Sourcemeter 2400 was used for electrical measurements. A voltage was applied to the sample, and the resulting current was determined. During these measurements, the current was limited to values below 1 μA, to avoid damage of the samples. Because the silver layer was sometimes damaged in the course of the experiment, at least four different samples were measured for each data point, and the lowest value was used. For a given batch of samples, the enhancement and the resulting resistance were reproducible. However, due to differences in surface activation and modification, samples prepared on different occasions exhibited the same qualitative phenomenon, but differences in the quatitative behavior

For direct microscopic characterization of the enhancement effect, parts of the substrate with the adsorbed gold particles were protected by a piece of poly(dimethylsiloxane) (PDMS), a soft polymeric material known from microcontact printing FIG. 7c). This procedure allows the preparation of two adjacent areas, where one area is modified and the other is in the original state (Fritzsche W., Ermantraut E., Köhler, J. M. Scanning 1998, 20, 106-109). Scanning electron microscopy was conducted using a DSM 960 (Carl Zeiss, Germany); no metal coating was applied before imaging. For scanning force microscopy, a NanoScope Dimension 3100 (Digital Instruments, Santa Barbara, Calif.) was used in tapping mode in air.

Results and Discussion Particle Immobilization

The quantification of bioanalyte concentration is a key problem in a variety of applications. A connection between the newly developed nanoparticle-based detection schemes with an electrical measurement of particle concentration would fill a technological gap in the existing setup. Although optical methods are in development (Reichert I., Csaki A., Köhler J. M., Fritzsche W. Anal. Chem. 2000, 72, 6025-6029; Taton T. A., Mirkin C. A., Letsinger R. L. Science 2000, 289, 1757-60) they lack the speed and ease of electrical schemes. We propose and demonstrate here a first step in this direction: An electrical classification of the particle concentration, which is based on a surface immobilization of the particles. To ensure an unbiased result of the classification, the surface density of adsorbed particles has to reflect the solution concentration.

Experiments with beads immobilized by DNA-DNA interactions demonstrated the correlation between solution and surface concentration (Reichert J., Csaki A., Köhler J. M., Fritzsche W. Anal. Chem. 2000, 72, 6025-6029) providing the base for a chip-based measurement of the solution concentration.

Two different adsorption schemes were used. The first one is based on specific DNA-DNA interactions, by covering the surface with a DNA monolayer that exhibits a sequence complementary to the one at the colloids. The interaction is sequence specific, as demonstrated by optical and SFM measurements (Reichert J., Csaki A., Köhler J. M., Fritzsche W. Anal. Chem. 2000, 72, 6025-6029; Möller A., Csaki A., Köhler, J. M., Fritzsche, W. Nuci. Ac. Res. 2000, 28, e91). This specificity allows a high parallelization by using substrates with many electrode gaps, each with a different species of immobilized DNA, which in turn is complementary to a DNA molecule of interest. FIG. 7 shows examples of colloids immobilized by DNA-DNA interactions.

For another set of applications, the overall solution concentration of DNA-modified gold beads should be determined, independent of their specific sequence. In such cases a substrate with a good adsorption behavior for such beads is needed. Silicon oxide, activated by dry etching, was identified as a surface material with the required adsorption properties. Examples of activated silicon oxide surfaces with adsorbed DNA-modified beads are shown in FIG. 8.

Silver Enhancement of Gold Nanoparticles

An electrode structure with particles bound by specific DNA-DNA interaction was prepared in which a surface covered by colloidal particles with a diameter of 30 nm was observed. Scanning force microscopy was used for a more detailed characterization of particle adsorption referring to surface density and particle distribution (cf. FIGS. 7a and 7d).

After imaging, this sample was subjected to a silver enhancement. The enhancement occurred by deposition of silver from the solution onto particles or other structures of gold. A first look onto the particles after enhancement showed a significant increase in diameter and thereby an improved visibility (FIGS. 7b and 7c), pointing to sufficient access of the solution to the particle surface. A deposition solely due to the DNA layer was be ruled out by looking at the DNA-modified silicon oxide surface, which showed no sign of metal deposition (background).

On the other hand, the gold electrode structure showed also a significant increase in height. The enhancement was more pronounced at the gold electrodes than compared to the particles, pointing to higher enhancement efficiency, as expected for these pure gold surfaces compared to the DNA-modified gold surface of the particles.

A side-by-side comparison of particles before and after enhancement was very helpful to identify and avoid imaging artifacts in SEM and SFM. Therefore, a sample was prepared that exhibited adjacent regions of unmodified and modified particles, using a wet-masking technique. By application of this technique (outlined in FIG. 6b), a sample with silver-enhanced particles was compared to original particles by SEM-imaging and SFM. The enhanced particles revealed greater electron and topographic contrast, pointing to an increase in diameter.

Assuming a spherical shape, the average diameter of both classes of particles was determined using the height information from SFM images. The enhancement increased the particle height from about 30 nm to about 90 nm.

Achieving an Electrically Conductive Layer

A conductive layer was prepared by deposition of silver (silver enhancement) and could be used for an electrical detection of particles on a surface. Scanning force microscopy was used to monitor the buildup of such a layer by stepwise silver enhancement of surface-adsorbed particles. The starting point was a sample with particles adsorbed in the electrode gap, as shown in the SFM image in FIG. 7d. The particles were normally separated from each other without significant clustering. They showed a typical height of about 25-30 nm; the electrical conductivity measured for this sample was below the detection limit.

After a first enhancement, the particles showed a clearly visible increase in diameter (FIG. 7e). The typical height was now about 90-120 nm. There was no complete conductive layer connecting the electrodes, as evident from SFM images and electrical measurements.

A second enhancement of the sample was performed (FIG. 7f). After this second enhancement, the whole substrate was covered with structures in the height of up to 300 nm. which was expected to result in a substantially more conductive layer. This assumption was confirmed by electrical measurements, which yielded resistivities of 5-10 Ohm. The surface of this sample was also examined using SEM, revealing a complete metal coverage of the electrode gap.

Electrical Measurements to Distinguish Particle Density

By using the enhancement procedure as a threshold parameter, one should expect to arrive at an enhancement protocol resulting in a conduction path for a higher density sample, but no conduction path for another, lower density. This approach for obtaining an electrical classification of the surface and thereby of the solution concentration was attempted.

A first realization of this approach was tested for the case of nonspecific binding (FIG. 8). Two samples exhibiting different surface densities were subject to a standardized enhancement protocol (see above). The resulting surfaces were observed by SFM and SEM. Both microscopic methods showed an interconnecting network in the case of the higher density sample and mostly individual particles without connections at the lower density sample. Therefore, it was concluded that it was possible to differentiate between samples of different densities by an electrical measurement. Such measurements resulted in resistivity of more than 200 Mohm for the low-density sample, compared to a value in the lower ohm range (below 10 Ohm) for the high-density sample. These resistivity differences of several magnitudes demonstrated the successful use of electrical measurements after a standardized enhancement to distinguish between different concentrations of DNA-modified beads.

Electrical Classification of Particle Density

An extended experiment was hereafter performed (See FIG. 9): A series of gold-particle labeled DNA solutions of 5 different concentrations (between 5.0 and 0.025 OD) was hybridized to DNA immobilized on surface substrates. Thereby, particles were now immobilized onto surfaces due to specific DNA-DNA interactions and DNA substrates with different surface densities of particles were obtained. These samples were enhanced by using the above described silver-enhancement procedure for different times, and the resistance was measured. The applied current was limited (to avoid destruction of the electrodes), resulting in a cutoff value of 500 Mohm. To address a whole range of different surface concentrations, the enhancement was conducted in a time series. Depending on the concentration and the enhancement time, a measurable decrease in resistance (from the cutoff value of 500 Mohm) was observed for all but the lowest value of particle concentration. Using the different times needed to achieve changes in the electrical resistance, a classification scheme could be established, which correlated time to concentration. In the case here described, a 2 min enhancement discriminates the highest concentration (5,0 OD) from the other samples, 8 min divides the samples in two groups, and 15 min discriminates the lowest concentration from the rest.

We demonstrated the electrical classification of particle concentration based on microelectrodes and metal enhancement. This approach works for unspecific adsorption, thereby allowing the application of the electrodes for a wide range of particles. On the other side, the use of highly specific molecular interactions, as demonstrated in the case of DNA, allows for a high specificity of electrodes prepared with special capture molecules. Therefore, the technique is suited for parallelization by using arrays of electrodes on one single chip, which can measure different target molecules. The use of electrical resistivity is straightforward, without the need for optical setups as in optical or fluorescent methods.

Other advantages are the easy signal processing and the potential for miniaturization. The invention is suited for quantification of the solution concentration of particles, and a parallel testing of different types of bioconjugated particles.

1. A method for detecting binding of members of a specific binding pair comprising providing a first member of said binding pair coupled to a deposition nucleus and specifically binding said first member to a surface-immobilized second member of said pair and determining the electrical resistance of said surface, the method characterized in that after binding of the members on said surface an electrically conductive deposit is formed on said surface under conditions that allow said deposit to be formed specifically on said nucleus or deposit formed.

2. A method according to claim 1, wherein said binding of said first and second member is realized by intermittent binding of associated binding partner.

3. A method according to claim 1, wherein said deposition nucleus comprises a surfaces that is catalytically active with regard to deposition of conductive deposits from solution.

4. A method according to claim 1, wherein said deposition nucleus comprises gold.

5. A method according to claim 1, wherein said deposit comprises silver.

6. A method according to claim 1, wherein said binding pair comprises nucleic acid, polypeptides, or derivatives and or analogues thereof.

7. A method according to claim 1, wherein said binding pair comprises nucleic acid, or derivatives and or analogues thereof.

8. A sensor comprising electrodes in contact with a surface comprising a binding pair wherein a member of said binding pair comprises a deposition nucleus and wherein said deposition nucleus comprises an electrically conductive deposit.

9. A sensor according to claim 8, wherein said nucleus comprises a surfaces that is catalytically active with regard to deposition of conductive deposits from solution.

10. A sensor according to claim 8, wherein said nucleus comprises gold.

11. A sensor according to claim 8, wherein said deposit comprises silver.

12. A sensor according to claim 8, wherein said binding pair comprises nucleic acid, polypeptides, or derivatives and or analogues thereof.

13. A sensor according to claim 8, wherein said binding pair comprises nucleic acid, or derivatives and or analogues thereof.

14. A method for quantifying a member of a specific binding pair in a sample comprising
linking the member in said sample with a deposition nucleus,
contacting a surface comprising an immobilized second member of said pair with said sample under conditions allowing specific binding of said pair,
subsequently performing an enhancement step on said surface by depositing an electrically conductive deposit on said nucleus and/or deposit formed, and
determining the electrical resistance of said surface and optionally comparing the electrical resistance of said surface with a reference.

15. A method according to claim 14, wherein said specific binding of said pair is realized by intermittent binding of associated binding partner.

16. A method according to claim 14, wherein said reference comprises a second surface on which said enhancement step was performed essentially in parallel and wherein said second surface comprises at known amount of said deposition nucleus.

17. A method according to claim 16, wherein said second surface comprises a known amount of first member comprising said deposition nucleus.

18. A method according to claim 17, wherein said first member was specifically bound to said immobilized second member essentially in parallel to said sample.

19. A method according to claim 14, wherein said reference comprises a set of surfaces wherein each surface comprises a different known amount of first member comprising said deposition nucleus, and on which said enhancement step was performed essentially in parallel with said sample.

20. A method according to claim 14, wherein at least one further enhancement step is performed.

21. A method according to claim 14, wherein said enhancement step is repeated until an electrical current is measured in said reference.
